(19) United States
(12) Patent Application Publication
(10) Pub. No.: US 2019/0225946 A1
(43) Pub. Date: Jul. 25, 2019

(54) RECOMBINANT MICROORGANISM HAVING ENHANCED ACTIVITY OF AT LEAST ONE OF 6-PHOSPHOGLUCONATE DEHYDROGENASE AND FOLDASE PROTEIN PrsA, AND USE THEREOF

(71) Applicants: Samsung Electronics Co., Ltd., Suwon-si (KR); Korea Advanced Institute of Science and Technology, Daejeon (KR)

(72) Inventors: Yukyung Jung, Hwaseong-si, Gyeonggi-do (KR); Taeyong Kim, Daejeon (KR); Byungkwan Cho, Daejeon (KR); Donghui Choe, Daejeon (KR); Seunghoon Song, Suwon-si, Gyeonggi-do (KR); Jinhwan Park, Suwon-si, Gyeonggi-do (KR)

(21) Appl. No.: 16/134,727

(22) Filed: Sep. 18, 2018

(30) Foreign Application Priority Data
Jan. 22, 2018 (KR) 10-2018-0007890

(51) Int. Cl. C12N 9/04; C12N 15/75; C02F 3/34; B01D 53/84; B01D 53/70; C12N 15/70; C12N 15/77; C12N 15/78; C12N 15/74

(52) U.S. Cl. CPC C12N 9/0006; C12N 15/75; C02F 3/34; B01D 53/84; B01D 53/70; C12Y 502/01008; C12N 15/77; C12N 15/78; C12N 15/743; C12Y 101/01044; C12N 15/70

(57) ABSTRACT

Provided is a recombinant microorganism having enhanced activity of at least one protein selected from 6-phosphogluconate dehydrogenase (6PGD) and foldase protein PrsA, a method of reducing a concentration of a fluorine-containing compound in a sample by using the recombinant microorganism, and a method of preparing the recombinant microorganism.

### CROSS-REFERENCE TO RELATED APPLICATION

This application claims the benefit of Korean Patent Application No. 10-2018-0007890, filed on Jan. 22, 2018, in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 9,002 Byte ASCII (Text) file named “739677_ST25.TXT,” created on Sep. 13, 2018.

### BACKGROUND

### 1. Field

The present disclosure relates to a recombinant microorganism having enhanced activity of at least one of 6-phosphogluconate dehydrogenase (6PGD) and foldase protein PrsA, a method of reducing a concentration of a fluorine-containing compound in a sample by using the recombinant microorganism, and a method of preparing the recombinant microorganism.

### 2. Description of the Related Art

One of the most serious environmental problems is the emission of greenhouse gases, which accelerate global warming. Accordingly, regulations aimed at reducing and preventing the emission of greenhouse gases have been tightened. Among the greenhouse gases, fluorinated gases (F-gases), such as perfluorocarbons (PFCs), hydrofluorocarbons (HFCs), and sulfur hexafluoride (SF₆), show low absolute emission. However, these gases have a long half-life and a very high global warming potential, resulting in significant adverse environmental impacts. The amount of F-gases emitted from the semiconductor and electronics industries, which are major sources of F-gas emissions, has already exceeded the assigned limits of greenhouse gas emissions, and yet, the amount of emissions still continues to increase. Therefore, the costs required to break down greenhouse gases and meet greenhouse gas emission allowances are increasing every year.

A pyrolysis or catalytic thermal oxidation process has generally been used for the decomposition of F-gases. However, this process has disadvantages, such as a limited decomposition rate, emission of secondary pollutants, and a high cost. Biological decomposition of F-gas using a microbial biocatalyst may overcome some of the limitations of the chemical decomposition process and allow F-gases to be treated in a more economical and environmentally friendly manner. Accordingly, there is a need to develop new microorganisms that decompose F-gas.

### SUMMARY

Provided herein is a recombinant microorganism comprising at least one genetic modification selected from a genetic modification that enhances 6-phosphogluconate dehydrogenase (6PGD) activity and a genetic modification that enhances foldase protein PrsA activity.

Also provided is a method of reducing a concentration of a fluorine-containing compound in a sample, the method comprising: contacting the recombinant microorganism with a sample containing a fluorine-containing compound, thereby reducing the concentration of the fluorine-containing compound in the sample.

Further provided is a method of preparing a recombinant microorganism having enhanced activity of removing a fluorine-containing compound from a sample containing the fluorine-containing compound.

Additional aspects of the invention are set forth in the detailed description that follows, and will be apparent from the description, or by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1 is a vector map of a pBBR122 vector;

FIG. 2 is a vector map of an expression vector comprising the gene encoding the 6PGD (gnd); and

FIG. 3 is a vector map of an expression vector comprising the gene encoding foldase protein (prsA).

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

The term “increase in activity”, or “enhanced activity”, as used herein, may refer to a detectable increase in the activity of a cell, a polypeptide, a protein, or an enzyme. The term “increase in activity” or “enhanced activity” may refer to an activity level of a modified (e.g., genetically engineered) cell, protein, or enzyme that is higher than that of a comparable cell, protein, or enzyme of the same type, such as a cell, protein, or enzyme that does not have a given genetic modification (e.g., the original or “wild-type” cell, polypeptide, protein, or enzyme).

The term “activity of a cell” may refer to activity of a specific protein or enzyme of the cell. For example, an activity level of the modified or engineered cell, protein, or enzyme may be increased by about 5% or greater, about 10% or greater, about 15% or greater, about 20% or greater, about 30% or greater, about 50% or greater, about 60% or greater, about 70% or greater, or about 100% or greater, as compared with that of an unmodified cell, protein, or enzyme of the same type, e.g., a wild-type cell, protein, or enzyme. An activity level of a specific protein or enzyme of a cell may be increased by about 5% or greater, about 10% or greater, about 15% or greater, about 20% or greater, about 30% or greater, about 50% or greater, about 60% or greater, about 70% or greater, or about 100% or greater, as compared with that of the same protein or enzyme of a parent cell, e.g., an non-engineered cell. Enhanced activity of a protein or enzyme is measured by using a method known to those of ordinary skill in the art.

An increase in activity of an enzyme or polypeptide can be achieved by an increase in the expression or specific activity thereof. The increase in the expression can be achieved by introduction of a polynucleotide encoding the enzyme or polypeptide into a cell, by otherwise increasing the copy number of the polynucleotide in the cell, or by a mutation in the regulatory region of the polynucleotide. A microorganism into which the gene is introduced may include a copy of the gene endogenously, or the cell may not include the gene prior to introduction of the polynucleotide. The gene can be operably linked to a regulatory sequence that allows expression thereof, for example, a promoter, an enhancer, a polyadenylation region, or a combination thereof.

A polynucleotide that is introduced into the cell, or a polynucleotide the copy number of is otherwise increased, may be endogenous or heterologous to the cell. The term “endogenous gene” refers to a gene that is already within a microorganism prior to introducing the genetic modification. The term “exogenous gene” refers to a gene that is introduced into a cell, and is, for example, homologous or heterologous with respect to the host cell into which the gene is introduced. The term “heterologous” refers to a gene that is “foreign,” or “not native”.

An “increase in copy number” of a gene can be caused by amplification of a gene or an introduction of a gene. An increase in copy number of a gene may be achieved by genetically engineering a cell to introduce an exogenous gene that does not exist in the non-engineered cell, or by introducing another copy of a gene already within the cell. The introduction of the gene may be mediated by a vehicle such as a vector. In one embodiment, the introduction is a transient introduction in which the gene is not integrated into the genome. In another embodiment, the introduction results in integration of the gene into the genome. The introduction may be performed, for example, by introducing a vector into the cell, wherein the vector includes a polynucleotide encoding a target polypeptide, and replicating the vector in the cell. The introduction may also be performed by integrating the polynucleotide into the genome.

The introduction of the gene may be performed by any known method, such as transformation, transfection, and electroporation. The gene may be introduced via a vehicle or vector, or may be introduced by itself. The vehicle or vector includes, for instance, a nucleic acid molecule or construct (e.g., cassette) that is capable of delivering other nucleic acids linked thereto. Examples of the vector include a plasmid, a virus-derived vector, or the like. A plasmid is a circular double-stranded DNA molecule linkable with another DNA. Examples of a virus expression vector include a replication-defective retrovirus, adenovirus, adeno-associated virus, or a combination thereof.

As used herein, the gene manipulation is performed by molecular biological methods known in the art.

The term “parent cell” refers to an original cell, e.g., a non-genetically engineered cell of the same type as the engineered microorganism. With regard to a specific genetic modification, the parent cell is a cell that lacks the specific genetic modification, but is identical in all other respects to the cell with the genetic modification. Thus, the parent cell is a cell that may be used as a starting material to produce a genetically engineered cell having enhanced activity of a given protein (e.g., a protein having about 85% or greater sequence identity to 6-phosphogluconate dehydrogenase or foldase protein PrsA). The same comparison applies to different genetic modifications.

The term “gene” as used herein refers to a polynucleotide that encodes a specific protein. A gene may include regulatory sequences, such as a 5′ non-coding sequence and/or a 3′ non-coding sequence, or may be free of regulatory sequences.

The term “sequence identity” of a polynucleotide (nucleic acid) or polypeptide, as used herein, refers to a degree of identity between nucleotide bases or amino acid residues of sequences over a particular region. The sequence identity is a value measured by comparing two sequences in specific comparable regions via optimal alignment of the two sequences. A percentage of sequence identity is calculated by, for example, comparing two optimally aligned sequences in the entire comparable region, determining the number of locations where the two sequences have an identical amino acid or identical nucleic acid—in order to obtain the number of matching locations, dividing the number of matching locations by the total number of locations in the comparable region (that is, a range size), and multiplying the result by 100 to obtain the percentage of the sequence identity. The percentage of the sequence identity may be determined using a known sequence comparison program, for example, BLASTN (NCBI), BLASTP (NCBI), CLC Main Workbench (CLC bio), MegAlign™ (DNASTAR Inc), etc. Unless indicated otherwise, parameters used in the program are defined as follows: Ktuple=2, Gap Penalty=4, and Gap length penalty=12.

The term “genetic modification” as used herein includes an artificial alteration in the constitution or structure of the genetic materials of a cell.

A “fluorine-containing compound” as used herein may refer to a compound of Formula 1 or Formula 2:

C(R₁)(R₂)(R₃)(R₄) <Formula 1>

(R₅)(R₆)(R₇)C—[C(R₁₁)(R₁₂)]ₙ—C(R₈)(R₉)(R₁₀). <Formula 2>

In in Formula 1 and 2, n may be an integer from 0 to 10.

R₁, R₂, R₃, and R₄ may each independently be F, Cl, Br, I, or H. In one embodiment, at least one of R₁, R₂, R₃, and R₄ is F.

R₅, R₆, R₇, R₈, R₉, R₁₀, R₁₁, and R₁₂ may each independently be F, Cl, Br, I, or H. In one embodiment, at least one of R₅, R₆, R₇, R₈, R₉, R₁₀, R₁₁, and R₁₂ is F. When n is 2 or greater, R₁₁ and R₁₂ may be the same as or different from one another.

Unless stated otherwise, percent composition (%) is expressed as w/w % based on the total weight of the composition.

According to one aspect of the invention, the recombinant microorganism may include at least one genetic modification selected from a genetic modification that enhances 6-phosphogluconate dehydrogenase (6PGD) activity and a genetic modification that enhances foldase protein PrsA activity (i.e., a genetic modification that enhances 6-phosphogluconate dehydrogenase (6PGD) activity, a genetic modification that enhances foldase protein PrsA activity, or both).

With regard to the recombinant microorganism, the microorganism may have an increase in enzyme activity of the 6PGD and/or foldase protein PrsA. 6PGD is an enzyme that catalyses 6-phosphogluconate into ribulose 5-phosphate in the presence of nicotinamide adenine dinucleotide phosphate (NADP). Foldase protein PrsA is an enzyme functioning to assist post-translational extracellular folding of secreted proteins. The foldase protein PrsA may have peptidyl-prolyl cis-trans isomerase activity.

The at least one genetic modification may be an increase in expression of at least one gene selected from a gene encoding 6PGD and a gene encoding foldase protein PrsA.

The at least one genetic modification may be an increase in the copy number of at least one gene selected from a gene encoding 6PGD and a gene encoding foldase protein PrsA.

In one embodiment, the 6PGD is an enzyme classified as EC 1.1.1.44, and the foldase protein PrsA is an enzyme classified as EC 5.2.1.8.

The 6PGD may have about 85% or greater, 90% or greater, or 95% or greater sequence identity to the amino acid sequence of SEQ ID NO: 1. The foldase protein PrsA may have about 85% or greater, 90% or greater, or 95% or greater sequence identity to the amino acid sequence of SEQ ID NO: 2.

The gene encoding 6PGD may have about 85% or greater, 90% or greater, or 95% or greater sequence identity to the nucleotide sequence of SEQ ID NO: 3. The gene encoding foldase protein PrsA may have about 85% or greater, 90% or greater, or 95% or greater sequence identity to the nucleotide sequence of SEQ ID NO: 4. With regard to the recombinant microorganism, the gene may be introduced into the microorganism by a general method known in the art, for example, transformation or electroporation.

The recombinant microorganism may have activity of reducing a concentration of a fluorine-containing compound in a sample. The fluorine-containing compound may be a compound represented by one of Formulae 1 and 2. The recombinant microorganism may belong to the genus Pseudomonas, the genus Xanthomonas, the genus Escherichia, the genus Agrobacterium, the genus Corynebacterium, the genus Bacillus, the genus Rhodococcus, the genus Mycobacterium, or the genus Klebsiella. The recombinant microorganism may be Pseudomonas saitens having an Accession No. KCTC 13107BP. The recombinant microorganism belonging to the genus Escherichia may be Escherichia Coli. The recombinant microorganism belonging to the genus Bacillus may be Bacillus bombysepticus SF3 microorganism (having an Accession No. KCTC 13220BP).

The recombinant microorganism may include one or more, for example, 2 or more, 3 or more, 4 or more, 5 or more, 10 or more, or 50 or more exogenous genes encoding the 6PGD and foldase protein PrsA. When a plurality of genes are included in the recombinant microorganism, the plurality of genes may be different from each other. In one embodiment, the gene is integrated into the genome of the recombinant microorganism. In another embodiment, the gene is independent of the recombinant microorganism's genome.

The recombinant microorganism may reduce a concentration of the fluorine-containing compound represented by one of Formulae 1 and 2 in a sample.

The concentration of the fluorine-containing compound may be reduced by introducing a hydroxyl group to carbon of the fluorine-containing compound by action of the protein on the C—F or C—H bond thereof, or by accumulating the fluorine-containing compound inside the cell of the microorganism. Further, the fluorine-containing compound may be reduced by cleavage of the C—F bond of the fluorine-containing compound, by conversion of the fluorine-containing compound into a different compound or material (e.g., degrading the fluorine-containing compound, or by intracellular accumulation of the fluorine-containing compound. The sample may be in a liquid or gas state. The sample may be industrial waste water or waste gas. The sample may be any material that includes the fluorine-containing compound. The fluorine-containing compound may be CF₄, CHF₃, CH₂F₂, CH₃F, or a mixture thereof. The recombinant microorganism may reduce the fluorine-containing compound at a greater rate than the parent cell.

According to another aspect of the invention, provided is a composition for reducing a concentration of a fluorine-containing compound represented by one of Formulae 1 and 2 in a sample, wherein the composition may include a recombinant microorganism including at least one genetic modification selected from a genetic modification that may enhance 6PGD activity and a genetic modification that may enhance foldase protein PrsA activity; and a diluent or a medium, wherein the recombinant microorganism may have increased activities of at least one of these proteins, as compared with a parent strain.

With regard to the composition, the recombinant microorganism, the sample, and the fluorine-containing compound may be the same as those described above.

With regard to the composition, the term “reducing” includes a decrease of a concentration of a fluorine-containing compound in a sample by any amount, and includes complete removal of the fluorine-containing compound from the sample. The sample may be a gas or a liquid. In one embodiment, the sample does not include the microorganism. In another embodiment, the composition may further include a material that increases solubility of the fluorine-containing compound for a medium or a culture medium.

The composition may be used to reduce the concentration of fluorine-containing compound in the sample by contacting the composition with the sample. The contacting may be performed in a liquid or solid phase. The contacting may be performed, for example, by contacting a culture medium in which the recombinant microorganism is being cultured with the sample. The culturing may be performed in a closed container for an extended period (e.g., one or more days, weeks, or months). The culturing may be performed under conditions in which the recombinant microorganism is allowed to proliferate. The conditions may include providing a medium, oxygen amount, agitation and temperature that is suitable for the growth of the host cell. The medium may contain one or more carbon sources, one or more nitrogen sources, and other nutrients.

According to another embodiment, provided is a method of reducing a concentration of a fluorine-containing compound in a sample, wherein the method may include contacting a recombinant microorganism including at least one genetic modification selected from a genetic modification that may enhance 6PGD activity and a genetic modification that may enhance foldase protein PrsA activity with a sample containing a fluorine-containing compound represented by one of Formulae 1 and 2, thereby reducing a concentration of the fluorine-containing compound in the sample.

With regard to the method, the recombinant microorganism and the sample containing a fluorine-containing compound may be the same as those described above.

With regard to the method, the contacting may be performed in a liquid or solid phase. The contacting may be performed, for example, by contacting a culture medium in which the recombinant microorganism is being cultured with the sample. The culturing may be performed under conditions in which the recombinant microorganism is allowed to proliferate. The contacting may be performed in an airtight closed container. The contacting may be performed during an exponential phase or a stationary phase of a growth stage of the recombinant microorganism. The culturing may be performed under aerobic or anaerobic conditions. The contacting may be performed in an airtight closed container under conditions in which the recombinant microorganism may survive or be viable. The conditions in which the recombinant microorganism may survive or be viable refer to conditions in which the recombinant microorganism is allowed to proliferate or remain in a resting state.

With regard to the method, the sample comprising the fluorine-containing compound may be in a liquid or gas phase. The sample may be industrial waste water or waste gas. The sample may be passively or actively contacted with the culture of the microorganism. The term ‘passive contacting’ refers to a contacting without an external driving force and the term ‘active contacting’ refers to a contacting with an external driving force. The sample may be, for example, sparged into the culture of the microorganism. That is, the sample may be sparged into a medium or a culture medium. The sparging may be sparging of the sample from the bottom to the top of the medium or the culture medium. The sparging may include injecting droplets of the sample.

With regard to the method, the contacting may be performed in a batch or continuous manner. The contacting may include, for example, contacting a sample comprising a fluorine-containing compound with a fresh recombinant microorganism, thereby reducing the concentration of the fluorine-containing compound in the sample, wherein the recombinant microorganism has a genetic modification that may enhance 6PGD activity and enhance foldase activity, as compared with a parent strain of the recombinant microorganism. Contacting the sample with the fresh recombinant may be performed repeatedly, such as twice or more, for example, three times, five times, or ten times or more times. The contacting may be continued or repeated until the concentration of the fluorine-containing compound in the sample reaches a desired reduced concentration.

According to a further embodiment, provided is a method of preparing a recombinant microorganism having enhanced activity of removing a fluorine-containing compound represented by one of Formulae 1 and 2 from a sample containing the fluorine-containing compound, wherein the method may include introducing at least one gene selected from a gene encoding 6PGD and a gene encoding foldase protein PrsA to a microorganism, wherein the recombinant microorganism belongs to the genus Pseudomonas, the genus Xanthomonas, the genus Escherichia, the genus Agrobacterium, the genus Corynebacterium, the genus Bacillus, the genus Rhodococcus, the genus Mycobacterium, or the genus Klebsiella:

The gene encoding 6PGD and the gene encoding foldase protein PrsA, the fluorine-containing compound represented by one of Formulae 1 and 2, and the sample are the same as those described above.

According to another embodiment, provided is a 6PGD having about 85% or greater, 90% or greater, or 95% or greater sequence identity to the amino acid sequence of SEQ ID NO: 1; a foldase protein PrsA having about 85% or greater, 90% or greater, or 95% or greater sequence identity to the amino acid sequence of SEQ ID NO: 2; or a combination thereof.

According to another aspect of the invention, provided is a polynucleotide encoding 6PGD having about 85% or greater, 90% or greater, or 95% or greater sequence identity to the amino acid sequence of SEQ ID NO: 1 or foldase protein PrsA having about 85% or greater, 90% or greater, or 95% or greater sequence identity to the amino acid sequence of SEQ ID NO: 2.

The polynucleotide may have about 85% or greater, 90% or greater, or 95% or greater sequence identity to a nucleotide sequence of SEQ ID NO: 3 and may encode a protein having 6PGD activity. The polynucleotide may have about 85% or greater, 90% or greater, or 95% or greater sequence identity to a nucleotide sequence of SEQ ID NO: 4 and may encode a protein having foldase protein PrsA activity.

According to another embodiment of the invention, provided is a vector including the polynucleotide. The vector refers to a nucleotide sequence that is used in delivering a polynucleotide to a cell. The vector may include a cloning vector or an expression vector. The cloning vector may include a cloning region and a replication origin. The expression vector may include a regulatory sequence essential for the expression. The regulatory sequence may include a promoter, an enhancer, a terminator, a polyA sequence, and/or a ribosome binding site.

According to a further embodiment, a recombinant microorganism may be used to remove a fluorine-containing compound from a sample.

According to another embodiment, a recombinant microorganism may be used to reduce a concentration of a fluorine-containing compound in a sample.

According to another aspect of the invention, a method of reducing a concentration of a fluorine-containing compound in a sample may effectively reduce a concentration of a fluorine-containing compound in a sample.

According to an aspect of another embodiment, a recombinant microorganism having enhanced activity of removing a fluorine-containing compound in a sample containing the fluorine-containing compound may be prepared.

According to an aspect of another embodiment, 6PGD or foldase protein PrsA may be used in removing a fluorine-containing compound from a sample, wherein the 6PGD protein may have about 85% or greater, 90% or greater, or 95% or greater sequence identity to the amino acid sequence of SEQ ID NO: 1, and the foldase protein PrsA may have about 85% or greater, 90% or greater, or 95% or greater sequence identity to the amino acid sequence of SEQ ID NO: 2.

According to another embodiment, a polynucleotide encoding the 6PGD protein or foldase protein PrsA and a vector including the polynucleotide may be used in producing 6PGD or foldase protein PrsA.

Hereinafter, the present disclosure will be described in more detail with reference to Examples. However, these Examples are for illustrative purposes only, and the present invention is not intended to be limited by these Examples.

### Example 1: Decomposition of Fluorine-Containing Compound by Pseudomonas saitens Having an Accession No. KCTC 13107BP into which Gene Encoding 6PGD (Gnd) and Gene Encoding Foldase Protein (PrsA) are Introduced

1. Preparation of Pseudomonas saitens Strains into which Bacillus Bombysepticus SF3-Derived Gnd and prsA were Respectively Introduced

6PGD gnd and foldase protein PrsA of Bacillus bombysepticus SF3 (having an Accession No. KCTC 13220BP), which is a microorganism that naturally decomposes organofluorines, for example, CF₄, were selected as enzymes involved in decomposition of organofluorines.

B. bombysepticus SF3 was incubated overnight in a Luria-Bertani (LB) medium while being stirred at a temperature of 30° C. at a rate of 230 revolutions per minute (rpm). The genomic DNA thereof was isolated using a total DNA extraction kit (available from Invitrogen Biotechnology). Subsequently, a polymerase chain reaction (PCR) was performed using B. bombysepticus SF3 (having an Accession No. KCTC 13220BP) genome as a template. A primer set of SEQ ID NOs: 5 and 6 and a primer set of SEQ ID NOs: 7 and 8 were used to obtain the amplified gnd and the amplified prsA, respectively.

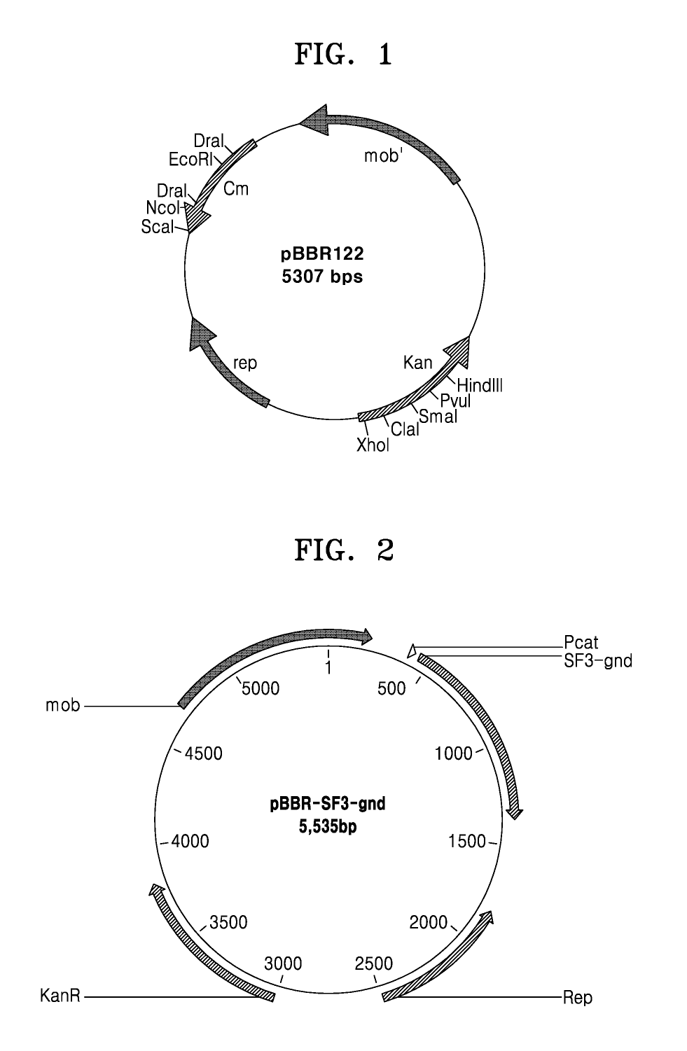

The amplified PCR products were cloned into separate pBBR122 vector fragments which were obtained by performing a PCR using pBBR122 vectors (available from MoBiTec GmbH, Germany) as a template and a primer set of SEQ ID NOs: 9 and 10, using InFusion Cloning Kit (available from Clontech Laboratories, Inc.). The pBBR122 vector developed by Dr. Camille from Pasteur Institute, France, is commercially available. The pBBR122 vector has a broad host range of bacteria. FIG. 1 shows a vector map of the pBBR122 vector.

As a result, a vector pBBR_SF3_gnd that expresses SF3-gnd gene and a vector pBBR_SF3_prsA that expresses SF3-prsA gene were obtained by inserting gnd and prsA into chloramphenicol resistance gene (cat) part of the pBBR122 vectors, respectively. SF3_gnd gene and SF3_prsA gene may each be expressed using a cat promoter (Pcat).

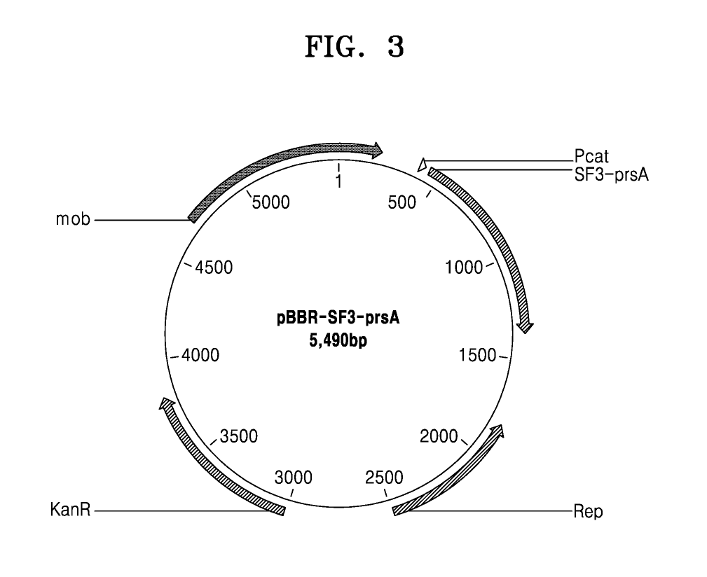

FIG. 2 shows a vector map of a gnd gene expression vector. FIG. 3 shows a vector map of a prsA gene expression vector.

Each of these vectors was introduced by electroporation to a Pseudomonas saitens (SF1) strain, which is a microorganism that naturally decomposes organofluorines (CF₄). Each strain was incubated in a LB plate medium including 50 micrograms per milliliter (μg/mL) of kanamycin to select the strain having kanamycin resistance. Each of the recombinant Pseudomonas saitens (SF1) strain was designated as SF1/pBBR_SF3_gnd and SF1/pBBR_SF3_prsA.

2. Decomposition of Fluorine-Containing Compound by Recombinant Pseudomonas saitens Strain

The two types of recombinant Pseudomonas saitens (SF1) strains prepared in Section 1, i.e., SF1/pBBR_SF3_gnd and SF1/pBBR_SF3_prsA, were tested their effects of removing CF₄ from a sample.

In detail, each of the two types of recombinant strains was incubated in an LB medium, while being stirred at a temperature of 30° C. and at a rate of 230 rpm, to harvest cells. The collected cells were suspended in a new LB medium at a cell density of OD₆₀₀=3.0. 20 milliliters (mL) of each cell suspension was added to a 50 mL-serum bottle, and the serum bottle was then airtight closed. The LB medium includes 10 grams per liter (g/L) of Tryptone, 5 g/L of yeast extract, and 10 g/L of NaCl.

Subsequently, gas-phase CF₄ was injected through a rubber stopper of a cap of the serum bottle using a syringe to its headspace at a concentration of 300 parts per million (ppm) Thereafter, the serum bottle was incubated for two days while being stirred at a temperature of 30° C. and at a rate of 230 rpm. Here, the experiment was performed in triplicate. Following the culture, a headspace concentration of CF₄ in the serum bottle was analyzed.

For analysis, 0.5 mL was collected from the headspace using a syringe and injected into a gas chromatography (GC) (Agilent 7890, Palo Alto, Calif., USA). The injected CF₄ was separated through a CP-PoraBOND Q column (25 meters (m) length, 0.32 millimeters (mm) i.d., 5 micrometers (μm) film thickness, available from Agilent), and changes in concentration of the separated CF₄ were analyzed by a Mass Selective Detector (MSD) (Agilent 5973, Palo Alto, Calif., USA). As a carrier gas, helium was applied to the column at a flow rate of 1.5 milliliters per minute (mL/min). GC conditions were as follows: an inlet temperature was 250° C., an initial temperature was maintained at 40° C. for 2 minutes, and temperature was raised to 290° C. at a rate of 20° C./min. Mass spectrometry (MS) conditions were as follows: ionization energy was 70 electron volts (eV), an interface temperature was 280° C., an ion source temperature was 230° C., and a quadrupole temperature was 150° C. As a control medium, the serum bottle not containing the cells was incubated under the same conditions at a CF₄ concentration of 300 ppm. Then, the amount of CF₄ gas was measured; however, there was no change in the amount.

Table 1 shows the results of decomposition rates of the two types of recombinant strains and the control measured under the same conditions of incubation, wherein the decomposition rate was measured by comparing the amounts of residual CF₄ in the samples with one another. As shown in Table 1, each of the recombinant Pseudomonas saitens strains, SF1/pBBR_SF3_gnd and SF1/pBBR_SF3_prsA, increased removal of CF₄ from the headspace 1.71 times greater and 1.49 times greater, respectively, as compared with the control strain to which an empty vector was introduced.

TABLE 1

| Recombinant P. saitens strain | CF₄ decomposition rate (%) |
|---|---|
| SF/pBBR122 (the control) | 6.13 |
| SF1/pBBR_SF3_gnd | 10.50 |
| SF1/pBBR_SF3_prsA | 9.13 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant microorganism comprising at least one genetic modification selected from a genetic modification that enhances 6-phosphogluconate dehydrogenase (6PGD) activity and a genetic modification that enhances foldase protein PrsA activity.

2. The recombinant microorganism of claim 1, wherein the at least one genetic modification is an increase in expression of at least one gene selected from a gene encoding 6PGD and a gene encoding foldase protein PrsA.

3. The recombinant microorganism of claim 1, wherein the at least one genetic modification is an increase in copy number of at least one gene selected from a gene encoding 6PGD and a gene encoding foldase protein PrsA.

4. The recombinant microorganism of claim 1, wherein the 6PGD is an enzyme classified as EC 1.1.1.44, and the foldase protein PrsA is an enzyme classified as EC 5.2.1.8.

5. The recombinant microorganism of claim 2, wherein the 6PGD and the foldase protein PrsA respectively have about 85% or greater sequence identity to the amino acid sequences of SEQ ID NO: 1 and SEQ ID NO: 2.

6. The recombinant microorganism of claim 2, wherein the gene encoding 6PGD and the gene encoding foldase protein PrsA respectively have about 85% or greater sequence identity to nucleotide sequences of SEQ ID NO: 3 and SEQ ID NO: 4.

7. The recombinant microorganism of claim 1, wherein the recombinant microorganism belongs to the genus Pseudomonas, the genus Bacillus, the genus Xanthomonas, the genus Escherichia, the genus Agrobacterium, the genus Corynebacterium, the genus Rhodococcus, the genus Mycobacterium, or the genus Klebsiella.

8. The recombinant microorganism of claim 1, wherein the recombinant microorganism is Pseudomonas saitens having an Accession No. KCTC 13107BP comprising at least one genetic modification selected from a genetic modification that enhances 6-phosphogluconate dehydrogenase (6PGD) activity and a genetic modification that enhances foldase protein PrsA activity.

9. A method of reducing a concentration of a fluorine-containing compound in a sample, the method comprising:
contacting a recombinant microorganism of claim 1 with a sample comprising a fluorine-containing compound, thereby reducing the concentration of the fluorine-containing compound in the sample, wherein the fluorine-containing compound is represented by Formula 1 or Formula 2:
C(R₁)(R₂)(R₃)(R₄) Formula 1
(R₅)(R₆)(R₇)C—[C(R₁₁)(R₁₂)]n-C(R₈)(R₉)(R₁₀) Formula 2
wherein, in Formula 1 and Formula 2, n is an integer from 0 to 10;
R₁, R₂, R₃, and R₄ are each independently fluorine (F), chlorine (Cl), bromine (Br), iodine (I), or hydrogen (H), wherein at least one of R₁, R₂, R₃, and R₄ is F;
R₅, R₆, R₇, R₈, R₉, R₁₀, R₁₁, and R₁₂ are each independently F, Cl, Br, I, or H, wherein at least one of R₅, R₆, R₇, R₈, R₉, R₁₀, R₁₁, and R₁₂ is F; and
when n is 2 or greater, R₁₁ and R₁₂ are identical to or different from each other.

10. The method of claim 9, wherein the at least one genetic modification is an increase in expression of at least one gene selected from a gene encoding 6PGD and a gene encoding foldase protein PrsA.

11. The method of claim 9, wherein the at least one genetic modification is an increase in copy number of at least one gene selected from a gene encoding 6PGD and a gene encoding foldase protein PrsA.

12. The method of claim 9, wherein the 6PGD is an enzyme classified as EC 1.1.1.44, and the foldase protein PrsA is an enzyme classified as EC 5.2.1.8.

13. The method of claim 9, wherein the 6PGD and the foldase protein PrsA respectively have about 85% or greater sequence identity to the amino acid sequences of SEQ ID NO: 1 and SEQ ID NO: 2.

14. The method of claim 9, wherein the contacting is performed in an airtight closed container.

15. The method of claim 9, wherein the contacting comprises culturing or incubating the recombinant microorganism while contacting the recombinant microorganism with the sample containing a fluorine-containing compound represented by one of Formula 1 and Formula 2.

16. The method of claim 9, wherein the contacting comprises culturing the recombinant microorganism in an airtight closed container under conditions in which the recombinant microorganism is allowed to proliferate.

17. The method of claim 9, wherein the fluorine-containing compound is CF₄, CHF₃, CH₂F₂, or CH₃F.

18. The method of claim 9, wherein the recombinant microorganism belongs to the genus Pseudomonas, the genus Bacillus, the genus Xanthomonas, the genus Escherichia, the genus Agrobacterium, the genus Corynebacterium, the genus Rhodococcus, the genus Mycobacterium, or the genus Klebsiella.

19. The method of claim 9, wherein the sample is in a liquid or gas state.

20. A method of preparing a recombinant microorganism of claim 1, the method comprising: introducing at least one gene selected from a gene encoding 6PGD and a gene encoding foldase protein PrsA into a microorganism, wherein the recombinant microorganism belongs to the genus Pseudomonas, the genus Bacillus, the genus Xanthomonas, the genus Escherichia, the genus Agrobacterium, the genus Corynebacterium, the genus Rhodococcus, the genus Mycobacterium, or the genus Klebsiella.


SEQUENCE LISTING (10 sequences)

SEQ ID NO: 1 - protein, 298 aa, Artificial Sequence
  Synthetic Bacillus bombysepticus SF3
MKLGLIGLGKMGFPLAEHLYEDKHEVVVYDVNKELVEKAGKLGITARHTLKEMIAELEAPRTIWVMVPAGEVVESVLKDVYPLLDEGDIVIEGGNSFYKDTLRRAEEAKSFGLHYVDIGTSGGVEGARYGACLMVGGEKEIYDQLEPLFKDLAVENGYSYAGRVGSGHFLKMVHNGIEYGMMQAIAEGFEVLDKSDFDFNYEDVAKVWANGSVIRGWLMDLTEKAFAEDPKLDGIKGVMNSSGEGKWTVETALELQAAAPVIAMSLFMRYRSQEDDTFHGKVVSALRNQFGGHEVVKK

SEQ ID NO: 2 - protein, 283 aa, Artificial Sequence
  Synthetic Bacillus bombysepticus SF3
MKKKKIFIGTIISCMMLALSACGSSDNVVTSKVGNVTEKELSKELRQQYGESTLYQMMLSKALLDKYKVSDEEAKKKVEEAKDKMGENFKSTLEQLGLKNEDELKEKMKPEIAFEKAIKATVTDKDVKDNYKPEMKVSHILVKDEKTAKEIKEKVNNGEDFAALAKQYSEDTGSKEQGGEISGFAPGQTVKEFEEAAYKLDAGQVSEPVKTTYGYHIIKVTDKKELKPFDEVKDKIRKDIEQQRLQDTTGKWKQQVVNDLLKDADIKVNNKEFKDTFKFLEKK

SEQ ID NO: 3 - DNA, 897 bp, Artificial Sequence
  Synthetic Bacillus bombysepticus SF3
atgaaactaggtttaattggattaggaaaaatgggattcccattagctgaacacttatatgaagacaagcatgaagtagtagtatacgatgtaaataaagagcttgttgaaaaggcaggaaaactaggaattactgcacgtcatacattaaaagaaatgatcgctgaactagaagctcctcgtacaatttgggtaatggtgcctgcaggagaagttgtagagtcagtactaaaagatgtttatccattattagatgaaggtgatatcgttattgaaggcggaaattcattctataaagatacgttacgccgtgctgaagaagcaaaaagcttcggattacattatgtagatatcggtacatcaggcggtgtagaaggagctagatacggtgcttgcttaatggttggtggagaaaaagaaatttacgaccaattagaacctttatttaaagatttagcagtagaaaatggctattcttatgctggccgtgttggtagtggtcatttcttaaaaatggttcataacggtattgagtacggcatgatgcaagcaatcgctgaaggatttgaagtactagataagagtgactttgatttcaattatgaagatgttgcaaaagtatgggcgaacggatcagttatccgtggttggttaatggacttaactgaaaaagcatttgcagaagatccgaaactagatggcattaaaggtgtaatgaactcttcaggagaaggaaaatggactgttgaaactgcacttgaacttcaagctgcagcaccagtaatcgctatgtcattatttatgcgctatcgttctcaggaagatgatacattccatggaaaagtagtttcagcactacgtaatcagttcggtggacatgaagttgtgaaaaaataa

SEQ ID NO: 4 - DNA, 852 bp, Artificial Sequence
  Synthetic Bacillus bombysepticus SF3
atgaaaaagaagaaaatatttattggaacaattatttcatgcatgatgctagcattatctgcatgtggttcctcagacaacgtagtaacatcaaaagtaggaaatgttacagagaaagaattaagtaaagaattacgacaacaatatggagaaagtactttatatcaaatgatgttaagtaaggcattgctagataaatataaagtttcagatgaggaagcaaaaaagaaagtagaagaagcaaaagacaaaatgggtgaaaactttaaatcgactttagaacaacttgggttgaaaaatgaagatgaattaaaagaaaaaatgaagccagaaattgcatttgagaaagcaattaaagcaacagttacagataaagatgtgaaagataactataaaccagaaatgaaagtaagtcacattttagtgaaagatgaaaaaacggctaaagaaataaaggagaaagtaaataatggtgaagattttgctgccttagcaaaacagtattcagaagatactggttcaaaggaacagggcggggaaatatctggttttgcccctggacaaacagtgaaagaatttgaggaagctgcgtataaattagatgcaggccaagtaagtgagccggttaaaacaacttacggttaccatattattaaagtgacggataaaaaagaattgaagccatttgatgaagtaaaggataaaattcgtaaagatatagaacaacaaagactacaagatacgacaggtaaatggaaacaacaagtagtcaatgatttattgaaagatgctgatattaaagtgaataataaagaatttaaagatacatttaaatttctagaaaagaaataa

SEQ ID NO: 5 - DNA, 29 bp, Artificial Sequence
  Synthetic primer
gaagctaaaatgaaactaggtttaattgg

SEQ ID NO: 6 - DNA, 32 bp, Artificial Sequence
  Synthetic primer
aataactgccttttattttttcacaacttcat

SEQ ID NO: 7 - DNA, 29 bp, Artificial Sequence
  Synthetic primer
gaagctaaaatgaaaaagaagaaaatatt

SEQ ID NO: 8 - DNA, 32 bp, Artificial Sequence
  Synthetic primer
aataactgccttttatttcttttctagaaatt

SEQ ID NO: 9 - DNA, 23 bp, Artificial Sequence
  Synthetic primer
cattttagcttccttagctcctg

SEQ ID NO: 10 - DNA, 20 bp, Artificial Sequence
  Synthetic primer
aaggcagttattggtgccct